(19) United States
(12) Patent Application Publication
(10) Pub. No.: US 2015/0313754 A1
(43) Pub. Date: Nov. 5, 2015

(54) INFANT WARMER

(71) Applicant: North Carolina State University, Raleigh, NC (US)

(72) Inventors: Ethan M. Hamilton, Raleigh, NC (US); Percy R. Hooper, JR., Cary, NC (US)

(21) Appl. No.: 14/268,091

(22) Filed: May 2, 2014

(51) Int. Cl. A61F 7/00; A61F 7/08

(52) U.S. Cl. CPC A61F 7/0085; A61F 7/08; A61F 2007/0054; A61F 2007/0095

(57) ABSTRACT

A heat exchange assembly includes a heat exchanger panel upon which a person is supportable. The heat exchanger panel includes at least two interconnected layers of polymer material and at least one channel defined between the layers through which a heat exchange fluid may be circulated. The assembly further includes a heat pump operable to circulate the heat exchange fluid through the channel.

### FIELD OF THE INVENTION

The present invention relates to heat exchange devices, and more particularly to infant warmers.

### BACKGROUND OF THE INVENTION

Infant warmers are commonly used in hospitals to maintain an infant at a proper temperature. Conventional infant warmers often employ a radiant heater or lamp that radiates infrared heat energy upon the infant. A temperature sensor is typically placed on the infant to provide control feedback to the radiant heater. This type of conventional infant warmer has several drawbacks. Because the heater is positioned above the infant, it can obstruct access to the infant. In addition, if the temperature sensor is forgotten or improperly positioned, the heater may overheat the infant.

### SUMMARY OF THE INVENTION

The invention provides, in one aspect, a heat exchange assembly including a heat exchanger panel upon which a person is supportable. The heat exchanger panel includes at least two interconnected layers of polymer material and at least one channel defined between the layers through which a heat exchange fluid may be circulated. The heat exchange assembly further includes a heat pump operable to circulate the heat exchange fluid through the channel.

The invention provides, in another aspect, a heat exchanger panel upon which a person is supportable. The heat exchanger panel includes at least two interconnected layers of polymer material and at least one channel defined between the layers through which a heat exchange fluid may be circulated to heat or cool the person.

Other features and aspects of the invention will become apparent by consideration of the following detailed description and accompanying drawings.

### BRIEF DESCRIPTION OF THE DRAWINGS

FIG. 1 is a perspective view of an infant warmer in accordance with an embodiment of the invention.

FIG. 2 is an exploded view of a portion of the infant warmer of FIG. 1, illustrating a heat exchanger panel.

FIG. 3 is a perspective view of the heat exchanger panel of FIG. 2 in communication with a heat pump of the infant warmer of FIG. 1.

FIG. 4 is an exploded view of the heat exchanger panel of FIG. 2.

FIG. 5 is an enlarged perspective view of a portion of the heat exchanger panel of FIG. 2.

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting.

### DETAILED DESCRIPTION

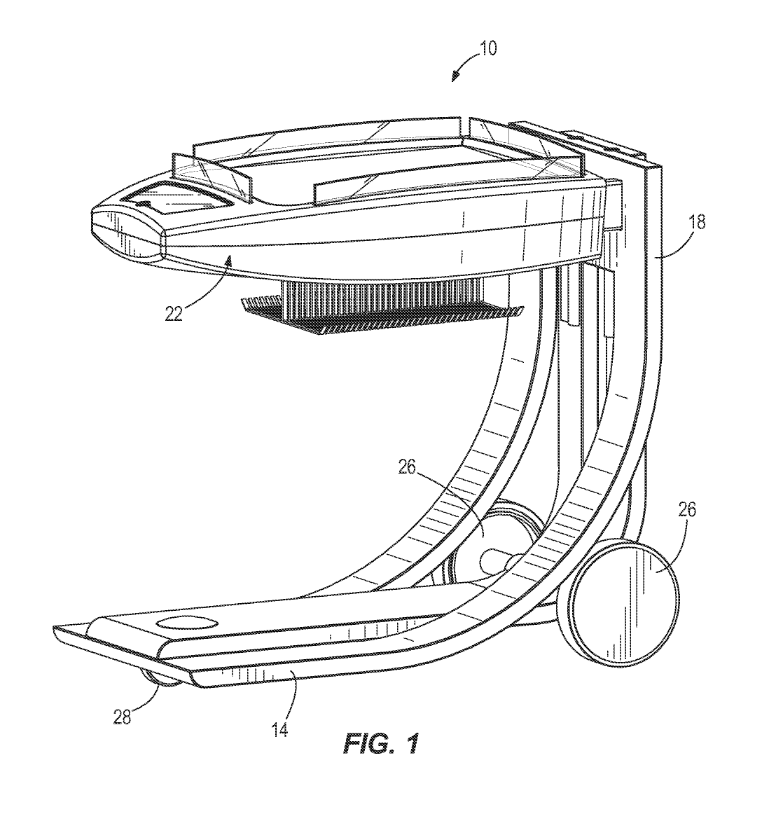

FIG. 1 illustrates an infant warmer 10 including a base 14, a support portion 18, and a warming platform 22 coupled to the support portion 18. In the illustrated embodiment, the warming platform 22 is cantilevered from the support portion 18 so that the warming platform 22 can be positioned over a hospital bed, for example. In other embodiments, the warming platform 22 can be supported in any other suitable fashion. Two rear wheels 26 and a recessed front caster 28 are coupled to the base 14 to facilitate maneuvering the infant warmer 10.

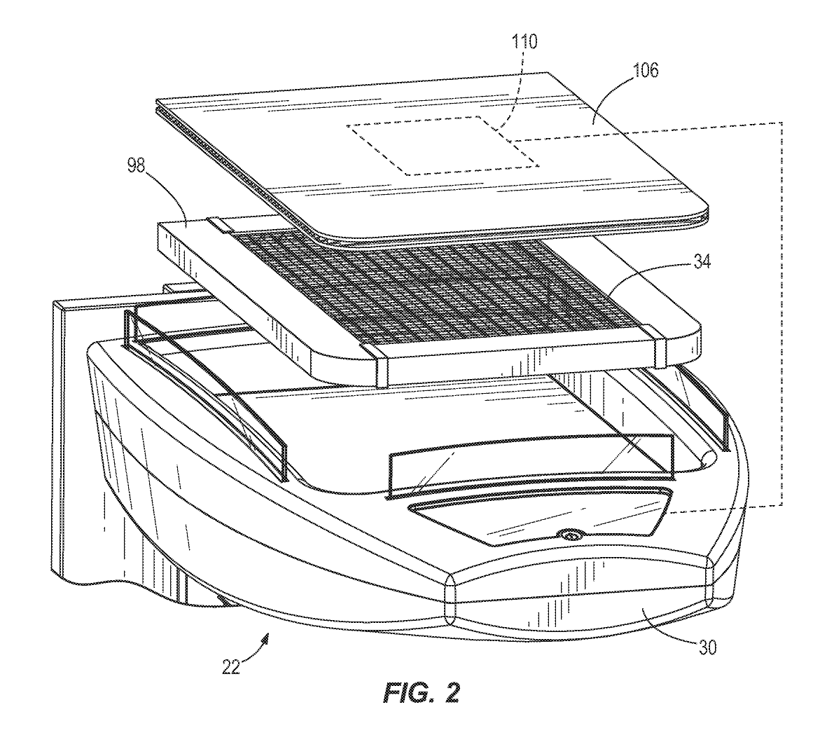

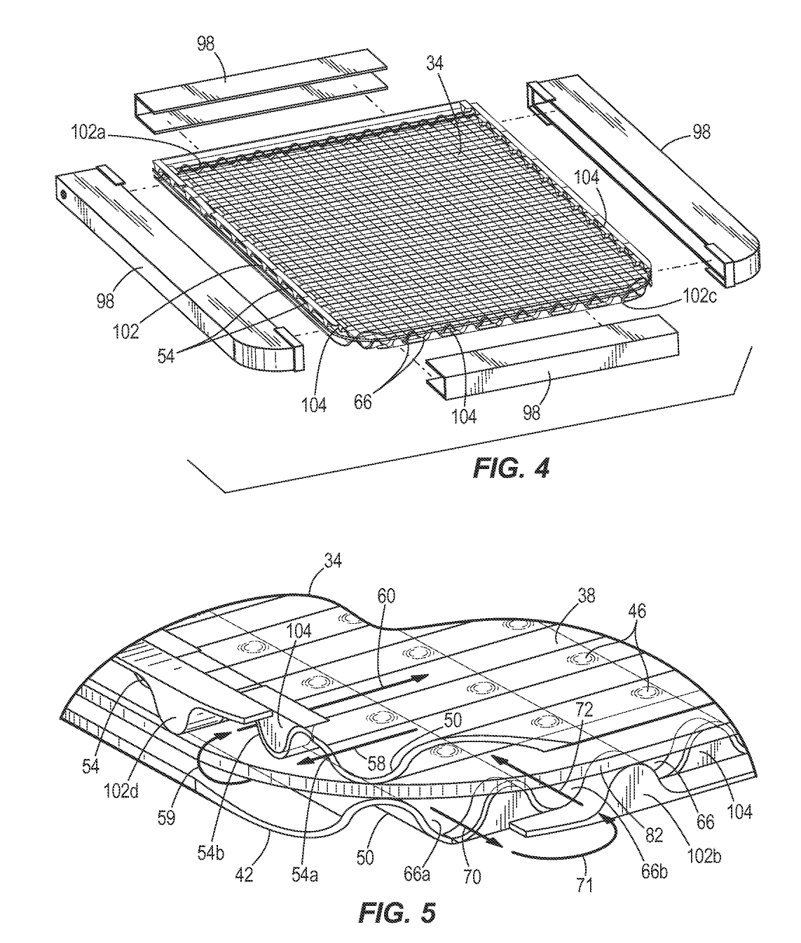

With reference to FIG. 2, the warming platform 22 includes a housing 30 and a heat exchanger panel 34, on which a person or infant is supportable, supported by the housing 30. Best illustrated in FIG. 5, the heat exchanger panel 34 is made of first and second interconnected sheets 38, 42 of a heat-shrinkable polymer material. The interconnected sheets 38, 42 may define a self-corrugating polymer panel such as those described in U.S. Patent Application Publication Nos. 2014/0087145, 2014/0087146, and 2014/0087147, the entire contents of all of which are incorporated herein by reference.

Before assembling the heat exchanger panel 34, each of the sheets 38, 42 is uniaxially stretched to impart direction or orientation in the polymer chains. The sheets 38, 42 are oriented so that the stretched direction of the first sheet 38 is generally perpendicular to the stretched direction of the second sheet 42. A radio frequency (RF) or other suitable welding process is used to create weld spots 46 that permanently bond the sheets 38, 42 at evenly-spaced intervals. The sheets 38, 42 are then heated, causing them to shrink along orthogonal axes. This creates undulations 50 in each of the sheets 38, 42 between adjacent weld spots 46.

With continued reference to FIG. 5, the undulations 50 in the first sheet 38 define a first plurality of channels 54 extending in a first direction 58, and the undulations 50 in the second sheet 42 define a second plurality of channels 66 extending in a second direction 70 that is orthogonal to the first direction 58. In the illustrated embodiment, the first and second channels 54, 66 are separated in a third direction 78, orthogonal to the first and second directions 58, 70, by a third sheet 82. Unlike the first and second sheets 38, 42, the third sheet 82 is not uniaxially stretched and therefore remains generally flat during the heat shrinking process described above.

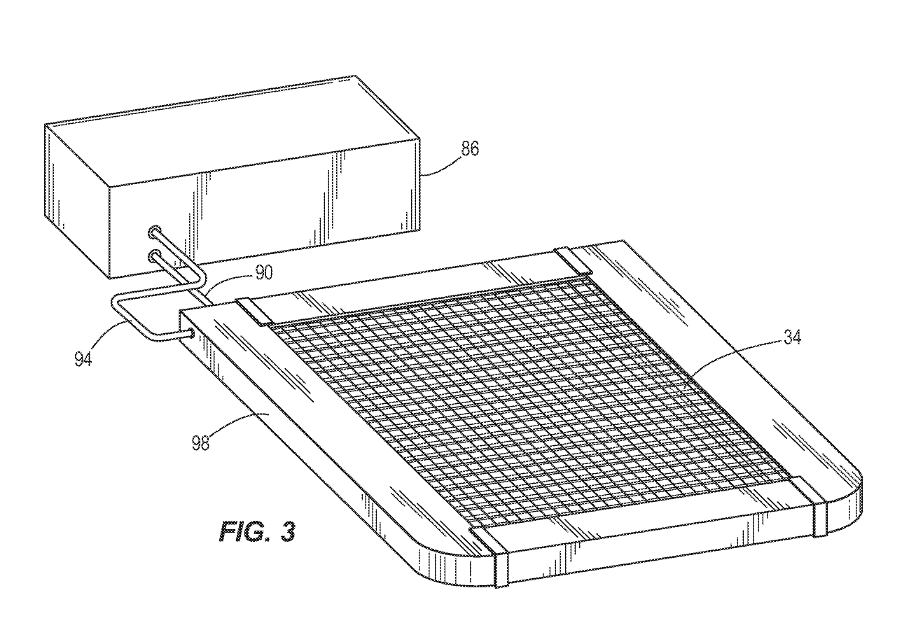

With reference to FIG. 3, the infant warmer 10 includes a digitally controlled heat pump 86 for circulating a heat exchange fluid (e.g., water) throughout the heat exchanger panel 34, thereby warming the heat exchanger panel 34 to a desired temperature set point. In the illustrated embodiment, the temperature set point may be between about 97.7 and about 99.5 degrees Fahrenheit (i.e. the body temperature of an infant). In other embodiments, the temperature set point may be any desired temperature below the glass transition temperature of the polymer sheets 38, 42, 82 and within the performance capabilities of the heat pump 86. In some embodiments, the heat pump 86 may be configured to remove heat from the heat exchanger panel 34 to provide a cooling effect.

Referring to FIGS. 3-5, the heat pump 86 fluidly communicates with the heat exchanger panel 34 via a supply line 90 and a return line 94 (FIG. 3). The supply line 90 and the return line 94 communicate with the respective first and second pluralities of channels 54, 66 to route the heat exchange fluid through the panel 34. In the illustrated embodiment, the supply line 90 feeds into a frame 98 surrounding the heat exchanger panel 34.

Once inside the frame 98, the fluid enters a first 66a of the channels 66 and flows through the channel 66a in the direction of arrow 70 (FIG. 5). The fluid is prevented from entering the adjacent channels 66 by a gasket 102a (FIG. 4) that seals against an interior of the frame 98. Accordingly, all of the supplied fluid enters the first channel 66a. When the fluid reaches the end of the panel 34 opposite the supply line 90, it exits the first channel 66a and enters the open end of a second channel 66b adjacent the first channel 66a, generally following the flow path illustrated by arrow 71 in FIG. 5. A gasket 102b seals against the interior of the frame 98 to prevent the fluid from flowing beyond the entrance to the second channel 66b. Upon entering the second channel 66b, the fluid flows in the direction of arrow 72. The fluid continues to flow through the remaining channels 66 in series, encountering additional gaskets 102 such that the fluid flows back and forth through the channels 66 to create a serpentine flow pattern. Plugs 104 cooperate with the undulations 58 between adjacent channels 66 to prevent the fluid from leaking out of the heat exchanger panel 34.

Once the fluid exits the last of the channels 66, it is directed into a first 54a (FIG. 5) of the channels 54 by a gasket 102c (FIG. 4) such that the fluid flows through the channel 54a in the direction of arrow 58 (FIG. 5). When the fluid reaches the end of the panel 34, it exits the first channel 54a and enters the open end of a second channel 54b adjacent the first channel 54a, generally following the flow path illustrated by arrow 59 in FIG. 5. A gasket 102d seals against the interior of the frame 98 to prevent the fluid from flowing beyond the entrance to the second channel 54b. Upon entering the second channel 54b, the fluid flows in the direction of arrow 60. The fluid continues to flow through the remaining channels 54 in series, encountering additional gaskets 102 such that the fluid flows back and forth through the channels 54 to create a serpentine flow pattern. The fluid is ultimately drawn out of the panel 34 through the return line 94 (FIG. 3).

In other embodiments, the heat exchanger panel 34 may be configured to have any other fluid flow pattern. For example, the frame 98 may function as a manifold to supply the heat exchange fluid to each of the channels 66 in parallel. Because the second channels 66 are orthogonal to the first channels 54, the flow of supply and return fluid forms a crossing pattern.

With reference again to FIG. 2, the illustrated infant warmer 10 includes a comfort layer or pad 106 positioned on top of the heat exchanger panel 34 to provide a relatively soft and compliant surface on which to support the infant. The pad 106 can be made of a thermally conductive fabric or foam to allow heat transfer between the infant and the heat exchanger panel 34. One or more sensors 110 may be incorporated in the pad for detecting the temperature of the infant supported thereon. This temperature data can be provided to the heat pump 86 for varying the temperature of the circulated heated fluid in accordance with the desired temperature set point.

In operation, an infant to be warmed is placed on the pad 106 on top of the heat exchanger panel 34. The heat pump 86 is then activated to begin supplying heated fluid (e.g., water) to the heat exchanger panel 34. The heated fluid is pumped through the supply line 90 and circulates through the channels 66, 54. Heat is transferred from the heated fluid flowing through the panel 34, through the pad 106, and to the infant. The fluid loses heat during this process and returns to the heat pump 86 through the return line 94 to be reheated. The one or more sensors 110 in the pad provide feedback to the heat pump 86 in order to reliably heat the infant to the desired temperature set point.

Although the heat exchanger panel 34 is described herein as part of an infant warmer 10, the heat exchanger panel 34 may be adapted for a variety of other applications. For example, the heat exchanger panel 34 may be incorporated into a mattress, automotive seat cushion, or any other application where controlled heating or cooling is desired.

Various features of the invention are set forth in the following claims.

What is claimed is:

1. A heat exchange assembly comprising:
a heat exchanger panel upon which a person is supportable, the heat exchanger panel including
at least two interconnected layers of polymer material, and
at least one channel defined between the layers through which a heat exchange fluid may be circulated; and
a heat pump operable to circulate the heat exchange fluid through the channel.

2. The heat exchange assembly of claim 1, wherein the layers are interconnected by a plurality of weld spots located at spaced intervals.

3. The heat exchange assembly of claim 2, wherein the layers include undulations between adjacent weld spots.

4. The heat exchange assembly of claim 3, wherein the channel is at least partially defined by the undulations.

5. The heat exchange assembly of claim 1, wherein the channel is a first channel extending in a first direction, and wherein the heat exchanger panel further includes a second channel extending in a second direction orthogonal to the first direction.

6. The heat exchange assembly of claim 5, wherein the first channel and the second channel are in fluid communication.

7. The heat exchange assembly of claim 6, wherein the heat exchanger panel further includes a gasket configured to redirect the heat exchange fluid from the first channel into the second channel.

8. The heat exchange assembly of claim 5, wherein the first channel is offset from the second channel in a third direction orthogonal to the first direction and the second direction.

9. The heat exchange assembly of claim 1, wherein the heat exchange fluid includes water.

10. The heat exchange assembly of claim 1, further comprising a pad disposed on top of the heat exchanger panel for supporting the person thereon.

11. The heat exchange assembly of claim 10, further comprising a temperature sensor coupled to the pad for detecting a temperature of at least one of the pad and the person supported thereon.

12. The heat exchange assembly of claim 11, wherein the heat pump includes a controller in communication with the temperature sensor for varying the temperature of the heat exchange fluid to approach a predetermined temperature set point.

13. The heat exchange assembly of claim 1, wherein the heat pump is operable to warm the heat exchanger panel to a temperature between about 97.7 degrees Fahrenheit and about 99.5 degrees Fahrenheit.

14. A heat exchanger panel upon which a person is supportable, the heat exchanger panel comprising:
at least two interconnected layers of polymer material; and
at least one channel defined between the layers through which a heat exchange fluid may be circulated to heat or cool the person.

15. The heat exchanger panel of claim 14, wherein the layers are interconnected by a plurality of weld spots located at spaced intervals.

16. The heat exchanger panel of claim 15, wherein the layers include undulations between adjacent weld spots.

17. The heat exchanger panel of claim 16, wherein the channel is at least partially defined by the undulations.

18. The heat exchanger panel of claim 14, wherein the channel is a first channel extending in a first direction, and wherein the heat exchanger panel further includes a second channel in fluid communication with the first channel, the second channel extending in a second direction orthogonal to the first direction.

19. The heat exchanger panel of claim 18, wherein the heat exchanger panel further includes a gasket configured to redirect the heat exchange fluid from the first channel into the second channel.

20. The heat exchanger panel of claim 18, wherein the first channel is offset from the second channel in a third direction orthogonal to the first direction and the second direction.
